SEQUENCE LISTING (10 sequences)

SEQ ID NO: 1 - protein, 10 aa, Artificial Sequence
  CDR-H1
GFTFTSTGIS

SEQ ID NO: 2 - protein, 18 aa, Artificial Sequence
  CDR-H2
GRIYPTSGSTNYADSVKG

SEQ ID NO: 3 - protein, 20 aa, Artificial Sequence
  CDR-H3
ARTYGIYDLYVDYTEYVMDY

SEQ ID NO: 4 - protein, 11 aa, Artificial Sequence
  CDR-L1
RASQDVDTSLA

SEQ ID NO: 5 - protein, 7 aa, Artificial Sequence
  CDR-L2
SASFLYS

SEQ ID NO: 6 - protein, 9 aa, Artificial Sequence
  CDR-L3
QQSTGHPQT

SEQ ID NO: 7 - protein, 123 aa, Artificial Sequence
  Heavy chain variable region
  MISC_FEATURE (26)..(35): CDR-H1
  MISC_FEATURE (49)..(66): CDR-H2
  MISC_FEATURE (97)..(116): CDR-H3
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLV

SEQ ID NO: 8 - protein, 108 aa, Artificial Sequence
  Light chain variable region
  MISC_FEATURE (24)..(34): CDR-L1
  MISC_FEATURE (50)..(56): CDR-L2
  MISC_FEATURE (89)..(97): CDR-L3
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKR

SEQ ID NO: 9 - protein, 457 aa, Artificial Sequence
  Heavy chain
  MISC_FEATURE (1)..(123): Heavy chain variable region
  MISC_FEATURE (26)..(35): CDR-H1
  MISC_FEATURE (49)..(66): CDR-H2
  MISC_FEATURE (97)..(116): CDR-H3
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Light chain
  MISC_FEATURE (1)..(108): Light chain variable region
  MISC_FEATURE (24)..(34): CDR-L1
  MISC_FEATURE (50)..(56): CDR-L2
  MISC_FEATURE (89)..(97): CDR-L3
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2018/0222983 A1
(43) Pub. Date: Aug. 9, 2018

(54) METHODS, COMPOSITIONS, AND KITS FOR TREATMENT OF CANCER

(71) Applicant: BIOCLIN THERAPEUTICS, INC., San Leandro, CA (US)

(72) Inventor: Jocelyn HOLASH, Alameda, CA (US)

(21) Appl. No.: 15/890,278

(22) Filed: Feb. 6, 2018

Related U.S. Application Data
(60) Provisional application No. 62/455,494, filed on Feb. 6, 2017.
(60) Provisional application No. 62/511,869, filed on May 26, 2017.

(51) Int. Cl. C07K 16/28; A61P 35/00; A61K 39/395; A61K 31/337

(52) U.S. Cl. CPC C07K 16/2863; A61P 35/00; A61K 39/3955; A61K 2039/505; C07K 2317/76; C07K 2317/565; C07K 2317/56; A61K 31/337

(57) ABSTRACT

Provided herein are the use of FGFR3 inhibitors and taxanes to treat solid and hematologic cancers, as well as compositions and kits comprising an FGFR3 inhibitor and a taxane.

### PRIORITY CLAIM

This application claims priority to U.S. Provisional Application No. 62/455,494, filed Feb. 6, 2017, and U.S. Provisional Application No. 62/511,869, filed May 26, 2017, the disclosures of which are incorporated herein in their entirety.

### BACKGROUND

The present application is directed to methods, compositions, and kits that utilize a combination of an FGFR3 inhibitor and a taxane to treat cancer, and to the use of FGFR3 inhibitors and taxanes in treating cancer and formulating medicaments for treating cancer.

### SUMMARY

Provided herein in certain embodiments are methods of treating a solid or hematologic cancer in a subject in need thereof comprising administering a therapeutically effective amount of an FGFR3 inhibitor and a therapeutically effective amount of a taxane. In certain embodiments, the FGFR3 inhibitor binds FGFR3. In other embodiments, the FGFR3 inhibitor binds a ligand for FGFR3. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is B-701. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Debio 1347, ARQ 087, JNJ-42756493, PRN-1371, TAS-120, INCB 54828, and BAY 1163877. In certain embodiments, the taxane is paclitaxel. In other embodiments, the taxane is an analog of paclitaxel, including for example docetaxel or cabazitaxel. In still other embodiments, the taxane is a prodrug of paclitaxel. In certain embodiments, the FGFR3 inhibitor and taxane are administered separately, i.e., in separate pharmaceutical formulations, either sequentially or simultaneously. In other embodiments, the FGFR3 inhibitor and taxane are administered together, i.e., in a single pharmaceutical formulation.

Provided herein in certain embodiments are compositions comprising an FGFR3 inhibitor and a taxane. In certain of these embodiments, the compositions are pharmaceutical formulations, and in certain embodiments these formulations comprise one or more pharmaceutically acceptable carriers. In certain embodiments, the FGFR3 inhibitor binds FGFR3. In other embodiments, the FGFR3 inhibitor binds a ligand for FGFR3. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is B-701. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Debio 1347, ARQ 087, JNJ-42756493, and PRN-1371, TAS-120, INCB 54828, and BAY 1163877. In certain embodiments, the taxane is paclitaxel. In other embodiments, the taxane is an analog of paclitaxel, including for example docetaxel or cabazitaxel. In still other embodiments, the taxane is a prodrug of paclitaxel.

Provided herein in certain embodiments are kits comprising an FGFR3 inhibitor and a taxane for use in treating cancer. In certain of these embodiments, the kits further comprise instructions for use.

Provided herein in certain embodiments are an FGFR3 inhibitor and a taxane for use in formulating a medicament for the treatment of cancer. In certain of these embodiments, the FGFR3 inhibitor and taxane are formulated into a single medicament. In other embodiments, the FGFR3 inhibitor and taxane are formulated into separate medicaments which are administered in combination with one another, either sequentially or simultaneously.

Provided herein are an FGFR3 inhibitor for use in co-administration with one or more taxanes to treat cancer, as well as a taxane for use in co-administration with one or more FGFR3 inhibitors to treat cancer.

### BRIEF DESCRIPTION OF DRAWINGS

FIG. 1: Kaplan-Meier plot showing survival of UM-UC-1 bladder cancer mice following administration of B-701, paclitaxel, and gemcitabine.

FIG. 2: Duration on treatment and survival for 19 Cohort 1 subjects administered B-701 and docetaxel.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

There are four single-pass transmembrane tyrosine kinase fibroblast growth factor receptors (FGFR1-4) in humans (Brooks 2012). FGFRs are overexpressed in many cancer types, often due to mutations that confer constitutive activation, making them an attractive target for therapeutic intervention. For example, the FGFR2b antibody FPA144 (FivePrime) is currently under development for the treatment of solid tumors, particularly gastric cancer. Other FGFR2 monoclonal antibodies in early development for cancer treatment include GP369 (Aveo) and HuGAL-FR21 (Galaxy) (Zhao 2010; Bai 2010). A humanized anti-FGFR4 has also been reported to inhibit tumor growth (Bumbaca 2011).

FGFR3 harbors both oncogenic and tumor suppressive properties. FGFR3 is frequently mutated or activated by gene fusion in certain cancers, e.g., urothelial cancers, non-small cell lung cancer (NSCLC), head and neck cancer, and glioblastoma, cell but in some normal tissues it can limit cell growth and promote cell differentiation (Lafitte 2013). The human FGFR3 antagonistic monoclonal antibody MFGR1877S (CAS No. 1312305-12-6), referred to herein as B-701 or BM2, was the first FGFR antibody to enter clinical development. B-701 is a lyophilized form of MGFR1877A. B-701 is currently in development for the treatment of metastatic bladder cancer (urothelial cell carcinoma) and achondroplasia (dwarfism). B-701 was originally identified through phage display, then recombined with a human IgG1 backbone. B-701 binds with high affinity to both wild-type and mutant FGFR3, including the most prevalent mutations found in bladder cancer and achondroplasia (specifically, FGFR3-IIIb^{R248C}, FGFR3-IIIb^{K652E}, FGFR3-III^{Y375C}, FGFR3-IIIb^{S249C}, and FGFR3-IIIb^{G373C}), while exhibiting no cross-reactivity with other FGFRs. B-701 was previously evaluated for safety in subjects with t(4:14) translocated multiple myeloma (Clinical Trial NCT01122875).

Other FGFR3 inhibitor antibodies currently in clinical or preclinical development include PRO-001 (Prochon) and IMC-D11 (ImClone). Additional FGFR3 antibodies for use in treating cancer and other diseases have been disclosed in, for example, U.S. Pat. Nos. 8,187,601 (Aveo) and 7,498,416 (Fibron).

It has previously been disclosed that administration of an FGFR3 antagonist antibody in combination with a programmed cell death protein (PD1) antagonist antibody unexpectedly resulted in slower tumor growth in mice than administration of either antibody alone (see, e.g., U.S. Patent Publ. No. 2016/0243228). These results were unexpected because FGFR3 antagonists and PD1 antagonists work at cross purposes with regard to immune function, with FGFR3 inhibitors having been shown previously to decrease immune response and PD1 inhibitors having been shown to upregulate T cell response.

As set forth in the Examples below, administration of an FGFR3 antagonist antibody in combination with the taxane paclitaxel in a mouse bladder cancer model resulted in an increase in survival that was significantly greater than that observed with administration of either agent alone. These results are surprising because cancers generally activate a number of survival pathways which blunt the effects of chemotherapy. Specifically blocking just one such pathway, FGFR3, would not be expected to profoundly enhance the effect of chemotherapy.

Subjects with locally advanced or metastatic urothelial carcinoma (UCC) have a poor prognosis. The standard treatment for UCC is administration of gemcitabine and cisplatin. In certain instances, subjects are further administered one or more immune checkpoint inhibitors. Until recently, there were no approved treatments for UCC subjects who progressed after receiving gemcitabine and cisplatin. Even with co-administration of immune checkpoint inhibitors, tumors in most of these subjects are unresponsive.

As further set forth in the Examples below, administration of an FGFR3 antagonist antibody in combination with the taxane docetaxel resulted in a significant increase in progression-free survival in human subjects with severe UCC who had previously received standard treatment. FGFR3 is highly expressed in urothelial carcinoma (UCC), and 15-20% of subjects with advanced disease have tumors with FGFR3 gene mutations or fusions. The observed increase in survival was most pronounced in subjects with such FGFR3 mutations or fusions. These results are surprising because previous studies have shown that a combination of taxane and FGFR inhibitor, specifically AZD4547 and docetaxel, was not tolerated in humans (ClinicalTrials.gov Clinical Identifier NCT01824901). Further, previous preclinical studies have shown that B-701 is efficacious in cancers expressing both wild-type and genetically activated forms of FGFR3 (Du 2011). The reported data demonstrate enhanced efficacy in tumors in which FGFR3 is genetically activated.

The present disclosure provides practical applications of the findings set forth herein in the form of compositions, methods, and kits for treating cancers, including solid cancers, using a combination of one or more FGFR3 inhibitors and one or more taxanes.

Provided herein in certain embodiments are methods of treating a solid or hematologic cancer in a subject in need thereof comprising administering an FGFR3 inhibitor and a taxane. Also provided herein are methods of increasing the effectiveness of a taxane for treating cancer in a subject in need thereof comprising administering an FGFR3 inhibitor or, conversely, methods of increasing the effectiveness of an FGFR3 inhibitor for treating cancer in a subject in need thereof comprising administering a taxane. An increase in effectiveness of a taxane or FGFR3 inhibitor may refer to an increase in the therapeutic effect of either agent, a decrease in the required dosage, administration frequency, or administration interval of either agent to obtain a particular level of therapeutic effect, or some combination thereof.

The term “solid cancer” as used herein refers to a cancer that forms a discrete tumor mass, i.e., a solid tumor. Examples of solid cancers within the scope of the present methods include cancers of the bladder, colon, rectum, kidney, prostate, brain, breast, liver, lung, skin (e.g., melanoma), and head and neck.

The term “hematologic cancer” as used herein refers to cancers that occur in cells of the immune system or in blood-forming tissues including bone marrow and which generally do not form solid tumors. Examples of hematologic cancers within the scope of the present methods include leukemia (e.g., acute myeloid leukemia, acute lymphoblastic leukemia, chronic myelogenous leukemia, and chronic lymphocytic leukemia), Hodgkin and non-Hodgkin lymphoma, myeloma, and myelodysplastic syndrome.

The terms “treat,” “treating,” and “treatment” as used herein with regard to solid cancers may refer to partial or total inhibition of tumor growth, reduction of tumor size, complete or partial tumor eradication, reduction or prevention of malignant growth, partial or total eradication of cancer cells, or some combination thereof. The terms “treat,” “treating,” and “treatment” as used herein with regard to hematological cancers may refer to complete or partial regression or remission, prevention, slowing, or reduction of cancer remission, partial or total eradication of cancer cells, or some combination thereof.

A “subject in need thereof” as used herein refers to a mammalian subject, preferably a human, who has been diagnosed with solid or hematologic cancer, is suspected of having solid or hematologic cancer, and/or exhibits one or more symptoms associated with solid or hematologic cancer. In certain embodiments, the subject may have previously received one or more therapeutic interventions for the treatment of cancer, e.g., chemotherapy. For example, in embodiments wherein the cancer being treated is bladder cancer, the subject may have previously been treated with gemcitabine and/or cisplatin. In certain embodiments wherein the subject has previously received one or more therapeutic interventions, the cancer being treated may have been refractory to intervention, with resistance occurring either at the outset of treatment or developing over time.

A “taxane” (also known as a “taxoid”) as used herein refers to paclitaxel (Taxol) or an analog or prodrug thereof. Taxanes are diterpene chemotherapeutic agents which function in part by disrupting microtubule function, resulting in inhibition of cell division. An “analog” of paclitaxel as used herein refers to a compound generated by replacing one or more atoms or functional groups of paclitaxel. The most well-known paclitaxel analog is the semi-synthetic analog docetaxel (Taxotere), which has been approved for treatment of a wide range of cancers, including lung cancer, breast cancer, and prostate cancer. Other paclitaxel derivatives include, but are not limited to, cabazitaxel (Jevtana), which is approved for treatment of prostate cancer, DJ-927 (Tesetaxel), XRP9881 (Larotaxel), BMS-275183, ortataxel, and RPR 109881A, and BMS-184476. A “prodrug” of paclitaxel as used herein refers to a compound that it is converted to paclitaxel following administration to a subject. Examples of paclitaxel prodrugs include, but are not limited to, DHA-paclitaxel (Taxoprexin) and paclitaxel polyglumex (Opaxio), both of which are in clinical development.

An “FGFR3 inhibitor” as used herein refers to any molecule that inhibits the activity of FGFR3 either partially or completely. An FGFR3 inhibitor may inhibit FGFR3 specifically, or it may inhibit the activity of other proteins in addition to FGFR3. For example, an FGFR3 inhibitor may also inhibit the activity of other FGFRs.

In certain embodiments of the methods, compositions, kits, and uses provided herein, the FGFR3 inhibitor inhibits FGFR3 activity by binding to FGFR3. Examples of such FGFR3 inhibitors include, for example, antagonistic FGFR3 antibodies or fusion proteins thereof, inactive forms of the FGFR3 ligand (e.g., truncated or otherwise mutated forms of the FGFR3 ligand) or fusion proteins thereof, small molecules, siRNAs, and aptamers. In certain of these embodiments, the FGFR3 inhibitor specifically binds FGFR3, meaning that the inhibitor exhibits little or no binding to other FGFRs. In other embodiments, the FGFR3 inhibitor binds one or more FGFRs in addition to FGFR3.

In certain preferred embodiments of the methods, compositions, kits, and uses provided herein, the FGFR3 inhibitor is an FGFR3 antagonist antibody, and in certain of these embodiments the FGFR3 antagonist antibody specifically binds FGFR3. The term “antibody” as used herein refers to an immunoglobulin molecule or an immunologically active portion thereof that binds to a specific antigen, e.g., FGFR3. In those embodiments wherein the FGFR3 antibody is a full-length immunoglobulin molecule, the antibody comprises two heavy chains and two light chains, with each heavy and light chain containing three complementary determining regions (CDRs). In those embodiments wherein the antibody is an immunologically active portion of an immunoglobulin molecule, the antibody may be, for example, a Fab, Fab′, Fv, Fab′F(ab′)₂, disulfide-linked Fv, scFv, single domain antibody (dAb), or a diabody. Antibodies for use in the present methods, compositions, kits, and uses may include natural antibodies, synthetic antibodies, monoclonal antibodies, polyclonal antibodies, chimeric antibodies, humanized antibodies, multispecific antibodies, bispecific antibodies, dual-specific antibodies, anti-idiotypic antibodies, or fragments thereof that retain the ability to bind a specific antigen, for example FGFR3. Exemplary antibodies include IgA, IgD, IgG1, IgG2, IgG3, IgM and the like. In certain preferred embodiments of the methods, compositions, kits, and uses provided herein, an FGFR3 antibody is an IgG2 antibody.

In certain embodiments, an FGFR3 antagonist antibody for use in the present methods, compositions, kits, and uses comprises a heavy chain variable region comprising one or more complementary determining regions (CDRs) having the sequences set forth in SEQ ID NOs:1-3. In certain of these embodiments, the FGFR3 antagonist antibody comprises all three of these CDR sequences, and in certain of these embodiments the FGFR3 antagonist antibody comprises a heavy chain variable region comprising the amino acid sequence set forth in SEQ ID NO:4. In certain embodiments, the FGFR3 antagonist antibody comprises a light chain variable region comprising one or more CDRs having the sequences set forth in SEQ ID NOs:5-7. In certain of these embodiments, the FGFR3 antagonist antibody comprises all three of these CDR sequences, and in certain of these embodiments the FGFR3 antagonist antibody comprises a light chain variable region comprising the amino acid sequence set forth in SEQ ID NO:8. In certain embodiments, the FGFR3 antagonist antibody comprises all six CDR sequences set forth in SEQ ID NOs:1-3 and 5-7, and in certain of these embodiments the FGFR3 antagonist antibody comprises the heavy chain variable region of SEQ ID NO:4 and the light chain variable region of SEQ ID NO:8. In certain embodiments, the antibody is B-701 comprising the heavy chain of SEQ ID NO:9 and the light chain of SEQ ID NO:10. In addition to the variable region set forth in SEQ ID NO:7, the heavy chain SEQ ID NO:9 comprises human IgG1. Similarly, the light chain of SEQ ID NO:10 comprises the variable region set forth in SEQ ID NO:8 and human Ig kappa chain C (UniProt P01834).

SEQ ID NO: 1 (H1-CDR):
GFTFTSTGIS.
SEQ ID NO: 2 (H2-CDR):
GRIYPTSGSTNYADSVKG.
SEQ ID NO: 3 (H3-CDR):
ARTYGIYDLYVDYTEYVMDY.
SEQ ID NO: 4 (L1-CDR):
RASQDVDTSLA.
SEQ ID NO: 5 (L2-CDR):
SASFLYS.
SEQ ID NO: 6 (L3-CDR):
QQSTGHPQT.
SEQ ID NO: 7:
EVQLVESGGGLVQPGGSLRLSCAASGFTFTSTGISWVRQAPGKGLEWVGR
IYPTSGSTNYADSVKGRFTISADTSKNTAYLQMNSLRAEDTAVYYCARTY
GIYDLYVDYTEYVMDYWGQGTLV.
SEQ ID NO: 8:
DIQMTQSPSSLSASVGDRVTITCRASQDVDTSLAWYKQKPGKAPKLLIYS
ASFLYSGVPSRFSGSGSGTDFTLTISSLQPEDFATYYCQQSTGHPQTFGQ
GTKVEIKR.
SEQ ID NO: 9:
EVQLVESGGGLVQPGGSLRLSCAASGFTFTSTGISWVRQAPGKGLEWVGR
IYPTSGSTNYADSVKGRFTISADTSKNTAYLQMNSLRAEDTAVYYCARTY
GIYDLYVDYTEYVMDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK.
SEQ ID NO: 10:
DIQMTQSPSSLSASVGDRVTITCRASQDVDTSLAWYKQKPGKAPKLLIYS
ASFLYSGVPSRFSGSGSGTDFTLTISSLQPEDFATYYCQQSTGHPQTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In other embodiments, an FGFR3 antagonist antibody for use in the present methods, compositions, kits, and uses may be PRO-001, IMC-D11, or an FGFR3 antagonistic antibody as disclosed in U.S. Pat. Nos. 8,187,601 (Aveo) or 7,498,416 (Fibron).

In certain embodiments of the methods, compositions, kits, and uses provided herein, the FGFR3 inhibitor inhibits FGFR3 activity by binding to a ligand for FGFR3. Examples of such FGFR3 inhibitors include, for example, antibodies that specifically bind an FGFR3 ligand or fusion proteins thereof, soluble forms of FGFR3 comprising all or part of the FGFR3 extracellular domain or fusion proteins thereof, truncated forms of FGFR3 lacking all or part of the intracellular domains required for downstream signaling or fusion proteins thereof, small molecules, siRNAs, and aptamers.

In certain embodiments of the methods, compositions, kits, and uses provided herein, the FGFR3 inhibitor is a pan-FGFR inhibitor, meaning that it binds to and inhibits the activity of one or more FGFRs in addition to FGFR3. In certain of these embodiments, the FGFR3 inhibitor may be a small molecule pan-FGFR inhibitor selected from the group consisting of infigratinib (BGJ398, Novartis), AZD4547 (AstraZeneca), LY2874455 (Eli Lilly), Debio 1347 (Debiopharm), ARQ 087 (ArQule), JNJ-42756493 (Janssen), PRN-1371 (Principia), TAS-120 (Taiho), INCB 54828 (Incyte), and BAY 1163877 (Bayer).

In certain embodiments of the methods, compositions, kits, and uses provided herein, the FGFR3 inhibitor inhibits FGFR3 activity by blocking downstream tyrosine kinase activity. For example, a non-selective tyrosine kinase inhibitor such as dovitinib, lucitinib, ponatinib, nintedanib, or ENMD-2076 may be utilized as an FGFR3 inhibitor.

In certain embodiments of the methods provided herein, the FGFR3 inhibitor and taxane are administered together, i.e., as part of the same pharmaceutical formulation. In other embodiments, the FGFR3 inhibitor and taxane are administered separately, i.e., in separate pharmaceutical formulations. In these latter embodiments, the agents may be administered simultaneously or sequentially, and may be administered via the same or different routes. In those embodiments wherein the agents are administered sequentially, they may be administered at the same or different intervals. For example, one agent may be administered more frequently than the other, or may be administered over a longer time course. In certain of these embodiments, one agent may be administered one or more times prior to the first administration of the second agent. When administration of the second agent is initiated, administration of the first agent may either cease or continue for all or part of the course of administration of the second agent. In certain embodiments wherein the agents are administered sequentially, the interval between administration of the first agent and administration of the second agent may be less than one minute, 1-5 minutes, 5-10 minutes, 10-30 minutes, 30-60 minutes, 1-2 hours, 2-4 hours, 4-6 hours, 6-12 hours, 12-24 hours, or more than 24 hours.

In certain embodiments of the methods provided herein wherein the FGFR3 inhibitor is an FGFR3 antagonist antibody, the antibody may be administered two or more times per day, daily, two or more times per week, weekly, bi-weekly (i.e., every other week), every third week, or monthly. In certain embodiments, the FGFR3 antagonist antibody is administered weekly, bi-weekly, or every third week. In certain embodiments, the FGFR3 antibody may be administered more frequently at or near the start of the treatment period. For example, the FGFR3 antibody may be administered daily, every 2-6 days, or weekly at the start of treatment, and then bi-weekly, every third week, or monthly for the remainder of the treatment period.

In certain embodiments of the methods provided herein, the taxane may be administered two or more times per day, daily, two or more times per week, weekly, bi-weekly, every third week, or monthly. In certain embodiments, the taxane is administered bi-weekly or every three weeks.

In certain embodiments of the methods provided herein, the FGFR3 inhibitor and/or taxane may be administered for a specific time course determined in advance. For example, the FGFR3 and/or taxane may be administered for a time course of 1 day, 2 days, 1 week, 2 weeks, 4 weeks, or 8 weeks. In other embodiments, the FGFR3 and/or taxane may be administered indefinitely, or until a specific therapeutic benchmark is reached. For example, the FGFR3 and/or taxane may be administered until tumor growth is arrested or reversed, until one or more tumors are eliminated, or until the number of cancer cells are reduced to a specific level.

A “therapeutically effective amount” of an agent as used herein is an amount of the agent that produces a desired therapeutic effect in a subject, such as treating cancer. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. The precise therapeutically effective amount for a particular agent will vary based on a variety of factors, including but not limited to the characteristics of the agent (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers present in the agent composition, and the route of administration. One skilled in the clinical and pharmacological arts will be able to determine a therapeutically effective amount through routine experimentation, namely by monitoring a subject's response to administration of the agent and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, N.Y., 2011, the entire disclosures of which are incorporated by reference herein.

In certain embodiments of the methods provided herein, a therapeutically effective amount of an FGFR3 inhibitor or taxane may be a dosage at which the agent is capable of generating a therapeutic response (e.g., reducing or eliminating tumor growth) as a monotherapy, i.e., when administered alone. In certain of these embodiments, the therapeutically effective amount may be a dosage that has previously been determined to be optimal or near optimal for cancer treatment. For example, where the FGFR3 inhibitor is B-701, the antibody may be administered at a dosage of about 10 to 50 mg/kg every two to four weeks, and in certain of these embodiments the antibody may be administered at a dosage of about 20 to 40 mg/kg every two to four weeks, or about 30 mg/kg every three weeks. In other embodiments, a therapeutically effective amount of an FGFR3 inhibitor or taxane may be lower than the dosage at which the agent would normally be administered for use as a monotherapy, i.e., a suboptimal dose. In certain of these embodiments, administration of the suboptimal dosage of FGFR3 inhibitor or taxane may result in decreased side effects versus the standard dosage when administered alone. For example, administration of suboptimal dosage of FGFR3 inhibitor or taxane may result in decreased occurrence or severity of pruritus, colitis, or pneumonia versus administration of the optimal dosage of either inhibitor alone. In certain embodiments, one of an FGFR3 inhibitor and a taxane may be administered at a dosage that has been determined to be optimal for cancer treatment when administered alone, while the other is administered at a dosage that is suboptimal for treatment when administered alone. In certain embodiments, the dosage of the FGFR3 inhibitor or taxane may change over the course of the treatment regimen. For example, one or both of the FGFR3 inhibitor and the taxane may be administered at higher dosage at the start of treatment (e.g., a loading phase), followed by a lower dosage later in treatment. In certain embodiments, this loading phase may also utilize more frequent administration than later phases of the treatment period.

An FGFR3 inhibitor, taxane, or pharmaceutical formulation comprising both an FGFR3 inhibitor and a taxane may be delivered to a subject by any administration pathway known in the art, including but not limited to parenteral, oral, aerosol, enteral, nasal, ophthalmic, parenteral, or transdermal (e.g., topical cream or ointment, patch). “Parenteral” refers to a route of administration that is generally associated with injection, including intravenous, intraperitoneal, subcutaneous, infraorbital, infusion, intraarterial, intracapsular, intracardiac, intradermal, intramuscular, intrapulmonary, intraspinal, intrasternal, intrathecal, intrauterine, subarachnoid, subcapsular, transmucosal, or transtracheal. In certain embodiments wherein the FGFR3 inhibitor is an FGFR3 antagonist antibody, including for example B-701, the FGFR3 inhibitor is administered intravenously. In certain embodiments, the taxane administered intravenously. In other embodiments, the taxane may be administered orally.

In certain embodiments of the methods, compositions, kits, and uses provided herein, the taxane is formulated for intravenous administration. In certain of these embodiments, the taxane is paclitaxel, and the intravenous formulation comprises Cremophor EL (CrEL) and dehydrated ethanol USP (1:1, v/v). In other embodiments, the taxane is docetaxel, and the intravenous formulation comprises polysorbate 80 (Tween 80). In other embodiments, the taxane is in a nanoparticle formulation. In certain of these embodiments, the taxane is nab-paclitaxel (Abraxane), a nanoparticle formulation approved for the treatment of several cancers including breast cancer and NSCLS in which paclitaxel is bound to human serum albumin, or polymeric-micellar paclitaxel (Genexol-PM), a formulation comprising biodegradable polymeric-micellar nanoparticles. In still other embodiments, the taxane is in a liposomal formulation. In certain of these embodiments, the taxane is EndoTAG-1, a cationic liposomal formulation of paclitaxel.

In certain embodiments, FGFR3 inhibitors, taxanes, or compositions comprising both FGFR3 inhibitor and taxane may be formed into oral dosage units, such as for example tablets, pills, or capsules. In certain embodiments, FGFR3 inhibitor, taxane, or FGFR3 inhibitor and taxane compositions may be administered via a time release delivery vehicle, such as, for example, a time release capsule. A “time release vehicle” as used herein refers to any delivery vehicle that releases active agent over a period of time rather than immediately upon administration. In other embodiments, FGFR3 inhibitor, taxanes, or FGFR3 inhibitor and taxane compositions may be administered via an immediate release delivery vehicle.

In certain embodiments of the methods provided herein, subjects receiving FGFR3 inhibitor and taxane may receive additional therapies, including for example additional chemotherapeutic agents or immunotherapy, before, during, or after treatment with FGFR3 and taxane. In certain of these embodiments, a subject may be further treated with a PD1 inhibitor, including but not limited to an antagonistic PD1 antibody (e.g., nivolumab (Opdivo®, pembrolizumab (Keytruda®), and MEDI-0680) or PD1 ligand antibody (e.g., atezolizumab (MPDL3280A, Tecentriq®), durvalumab (MEDI-4736), avelumab (MSB0010718C), RG7446, and BMS-936559). In those embodiments where the subject receives additional therapies during treatment with FGFR3 and taxane, the additional therapies may be administered simultaneously or sequentially with the FGFR3 inhibitor and/or taxane.

Provided herein in certain embodiments are pharmaceutical formulations comprising a therapeutically effective amount of an FGFR3 inhibitor and a therapeutically effective amount of a taxane. In certain embodiments, these pharmaceutical formulations further comprise one or more pharmaceutically acceptable carriers, or are formulated for administration with one or more pharmaceutically acceptable carriers. Also provided herein are kits comprising an FGFR3 inhibitor and a taxane for use in carrying out the methods disclosed herein, e.g., for treating cancer.

In certain embodiments of the compositions and kits provided herein, an FGFR3 inhibitor or taxane may be present in the composition or kit at a dosage at which it is capable of generating a therapeutic response (e.g., reducing or eliminating tumor growth) when administered alone. In certain of these embodiments, the FGFR3 or taxane may be present at a dosage that has previously been determined to be optimal or near optimal for cancer treatment. For example, where the FGFR3 inhibitor is B-701, the composition or kit may be formulated to deliver a dosage of about 10 to 50 mg/kg of B-701 to the subject, and in certain of these embodiments the composition or kit may be formulated to deliver a dosage of about 20 to 40 mg/kg or about 30 mg/kg of B-701 to the subject. In other embodiments, the FGFR3 inhibitor or taxane may be present at a dosage that is lower than that at which it would normally be present in a composition or kit for cancer treatment (i.e., a suboptimal dose).

A “pharmaceutically acceptable carrier” as used herein refers to a pharmaceutically acceptable material, composition, or vehicle that is involved in carrying or transporting a compound or molecule of interest from one tissue, organ, or portion of the body to another tissue, organ, or portion of the body. A pharmaceutically acceptable carrier may comprise a variety of components, including but not limited to a liquid or solid filler, diluent, excipient, solvent, buffer, encapsulating material, surfactant, stabilizing agent, binder, or pigment, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Examples of pharmaceutically acceptable carriers that may be used in conjunction with the compositions provided herein include, but are not limited to, (1) sugars, such as lactose, glucose, sucrose, or mannitol; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols such as propylene glycol; (11) polyols such as glycerin, sorbitol, mannitol and polyethylene glycol; (12) esters, such as ethyl oleate and ethyl laurate; (13) disintegrating agents such as agar or calcium carbonate; (14) buffering or pH adjusting agents such as magnesium hydroxide, aluminum hydroxide, sodium chloride, sodium lactate, calcium chloride, and phosphate buffer solutions; (15) alginic acid; (16) pyrogen-free water; (17) isotonic saline; (18) Ringer's solution; (19) alcohols such as ethyl alcohol and propane alcohol; (20) paraffin; (21) lubricants, such as talc, calcium stearate, magnesium stearate, solid polyethylene glycol, or sodium lauryl sulfate; (22) coloring agents or pigments; (23) glidants such as colloidal silicon dioxide, talc, and starch or tri-basic calcium phosphate; (24) other non-toxic compatible substances employed in pharmaceutical compositions such as acetone; and (25) combinations thereof.

Compositions and pharmaceutical formulations comprising an FGFR3 inhibitor, a taxane, or a combination of an FGFR3 inhibitor and a taxane may be formulated into a suitable dosage form, including for example solutions or suspensions in an aqueous or non-aqueous liquid, oil-in-water or water-in-oil liquid emulsions, capsules, cachets, pills, tablets, lozenges, powders, granules, elixirs or syrups, or pastilles. In certain embodiments, the compositions may be formulated as time release delivery vehicles, such as, for example, a time release capsule. A “time release vehicle” as used herein refers to any delivery vehicle that releases an active agent over a period of time rather than immediately upon administration. In other embodiments, the compositions may be formulated as immediate release delivery vehicles.

Provided herein in certain embodiments are kits for carrying out the methods disclosed herein. In certain embodiments, the kits provided herein comprise an FGFR3 inhibitor and a taxane. In certain embodiments, the FGFR3 inhibitor and taxane may be present in the kit in a single composition. In other embodiments, the FGFR3 inhibitor and taxane may be present in separate compositions. The kits may comprise additional therapeutic or non-therapeutic compositions. In certain embodiments, the kits comprise instructions in a tangible medium.

Provided herein in certain embodiments are an FGFR3 inhibitor and a taxane for use in the treatment of cancer. Also provided are an FGFR3 inhibitor for use in the treatment of cancer in combination with a taxane, and a taxane for use in the treatment of cancer in combination with an FGFR3 inhibitor.

Provided herein in certain embodiments is the use of an FGFR3 inhibitor and a taxane in the manufacture of a medicament for treating cancer. Also provided are the use of an FGFR3 inhibitor in the manufacture of a medicament for treating cancer in combination with a taxane, and the use of a taxane in the manufacture of a medicament for treating cancer in combination with an FGFR3 inhibitor.

The term “about” as used herein means within 10% of a stated value or range of values.

One of ordinary skill in the art will recognize that the various embodiments described herein can be combined. For example, steps from the various methods of treatment disclosed herein may be combined in order to achieve a satisfactory or improved level of treatment.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Effect of B-701, Paclitaxel, and Gemcitabine Administration on Tumor Growth and Survival

The effects of B-701, paclitaxel, and gemcitabine on tumor growth and survival were evaluated using the human UM-UC-1 bladder cancer cell line, which expresses wild-type FGFR3. Efficacy was assessed using tumor growth delay in a conditional survival study.

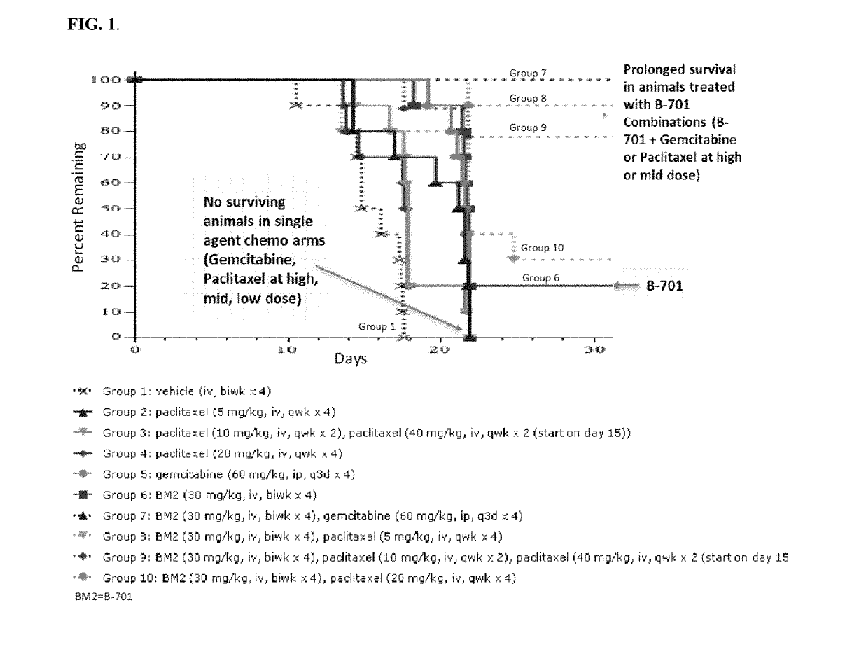

All three agents significantly extended survival of tumor bearing UM-UC-1 xenograft mice when administered as single agents. Administration of B-701 in combination with either paclitaxel or gemcitabine greatly enhanced this effect, resulting in a significant increase in conditional survival. These results are summarized in FIG. 1.

B-701, by blocking signaling through FGFR3, represents a novel and selective agent that can enhance the efficacy of both traditional and novel drugs currently being used to treat urothelial cancer. Preclinical models described here show that combining B-701 with chemotherapy leads to greatly enhanced efficacy.

### Example 2

### Effect of B-701 and Docetaxel Administration on Tumor Growth and Survival

In the first stage of a phase lb/2 study to evaluate the effects of B-701 and docetaxel in advanced UCC patients, safety and efficacy was evaluated in 19 human subjects (14 male, 5 female; “Cohort 1”) with stage IV UCC. Tumor characteristics for the 19 subjects are summarized in Table 1.

TABLE 1

| | | Homozygous |
|---|---|---|
| Subject | Known somatic variants/likely | deletions - |
| ID | functional rearrangements | known genes |
| 001-0001 | PIK3CA, TERT, TP53 | None |
| 001-0002 | ERBB2, TP53 | CDKN2A, CDKN2B |
| 001-0003 | None | None |
| 004-0004 | ERBB2, TERT, TP53 | None |
| 004-0005 | TERT, TP53 | None |
| 002-0006 | ERBB3, FGFR3,_S249C, | CDKN2A |
| | CREBBP_SLX4 | |
| 004-0007 | MST1R, PIK3CA, TERT | None |
| 002-0008 | FBXW7, NFE2L2, TERT | CDKN2A, CDKN2B |
| 004-0009 | Per investigator Oncopanel test: FGFR3_TACC3 | |
| 003-0010 | ARID1A, FGFR3_S249C, TERT, | LRP1B, TSC1, |
| | TP53 | CDKN2A, CDKN2B, |
| | KDM6A | |
| 005-0011 | ACVR1B, ERBB2, TERT, TP53, | None |
| | PBRM1_PBRM1_del | |
| 004-0012 | Subject progressed prior to treatment | |
| 004-0013 | PIK3R1 | CDKN2A, CDKN2B |
| 005-0014 | TP53 | None |
| 001-0015 | MLL2, PIK3CA, TERT | RB1 |
| 004-0016 | FGFR3_Y373C, MLL3, PIK3CA, | CDKN2A, CDKN2B |
| | TERT | |
| 005-0017 | FGFR3_S249C, PIK3CA, TERT, | CDKN2A |
| | SLIT2_SLIT2_del | |
| 004-0018 | ERB2, PIK3CA, TERT, TP53 | None |
| 004-0019 | ARID1A, BAP1, FANCI, RUNX1, | None |
| | TERT, FANCD2_FANCD2_trunc | |

| | |
|---|---|
| 005-0020 | To be determined |

All 19 subjects had an ECOG of 0 or 1, and had previously relapsed or were refractory to one or two prior non-taxane chemotherapeutic regimens. The median age of the subjects was 66 years. 11 subjects (58%) had an ECOG of 1, two subjects (11%) had a hemoglobin (Hgb) level less than 10 g/dL, five subjects (26%) had liver metastases, and 14 subjects (74%) had received two or more prior chemotherapeutic regimens.

Of the 19 subjects, five (4 male, 1 female, all marked with bold font in Table 1) had FGFR3 mutations or TACC3-fusions. The median age of this group was 65.4 years. Four of these five subjects (80%) had an ECOG of 1, one subject (20%) had liver metastases, and four subjects (80%) had received two or more prior chemotherapeutic regimens. None of the FGFR3 mutation/fusion subjects had an Hgb level less than 10 g/dL,

Docetaxel was administered intravenously over approximately 60 minutes at a dosage of 75 mg/m². B-701 was administered intravenously over approximately 90 minutes, and approximately 30 minutes after completion of docetaxel infusion, at a dosage of 25 mg/kg. Administration was carried out every 3 weeks (q3w). An additional loading dosage of 25 mg/kg B-701 was administered on day 8 of the first cycle. The primary objective was to evaluate progression-free survival (PFS) and safety. Secondary objectives included evaluation of overall response rate (ORR), duration of response (DOR), disease control rate (DCR), and overall survival (OS). Each of these objectives was evaluated for correlations with FGFR3 expression and/or FGFR3 mutations/fusions.

Treatment emergent adverse events (AEs) regardless of attribution that occurred in 10% of more of subjects (i.e., two or more subjects) are summarized in Table 2. Adverse effects grade 3 or higher related to B-701 that occurred in any subjects are summarized in Table 3.

TABLE 2

| Treatment emergent AE | # of subjects (%) |
|---|---|
| Diarrhea | 11 (58%) |
| Fatigue | 6 (32%) |
| Nausea | 6 (32%) |
| Alopecia | 5 (26%) |
| Constipation | 5 (26%) |
| Decreased neutrophil count | 5 (26%) |
| Fever | 5 (26%) |
| Decreased lymphocyte count | 4 (21%) |
| Abdominal pain | 3 (16%) |
| Anemia | 3 (16%) |
| Insomnia | 3 (16%) |
| Urinary tract infection | 3 (16%) |
| Decreased WBCs | 2 (11%) |
| Dysgeusia | 2 (11%) |
| Epistaxis | 2 (11%) |
| Headache | 2 (11%) |
| Hypotension | 2 (11%) |
| Neuropathy (peripheral) | 2 (11%) |
| Neutropenia | 2 (11%) |
| Pruritis | 2 (11%) |
| Upper respiratory tract infection | 2 (11%) |
| Vomiting | 2 (11%) |

TABLE 3

| Grade 3 or higher AE related to B-701 | # of subjects (%) |
|---|---|
| Decreased neutrophil count | 4 (21%) |
| Decreased WBCs | 2 (11%) |
| ALT elevation* | 1 (5%) |
| AST elevation* | 1 (5%) |
| Colitis | 1 (5%) |
| Decreased lymphocytes | 1 (5%) |
| Diarrhea | 1 (5%) |
| DIC | 1 (5%) |
| Fatigue | 1 (5%) |
| Hyponatremia | 1 (5%) |
| Infection (C. diff) | 1 (5%) |
| Thrombocytopenia | 1 (5%) |

*Resolved after docetaxel was discontinued

Four subjects had nine treatment-related AEs that led to B-701 dose interruption or modification. One subject discontinued treatment due to disseminated intravascular coagulation (DIC), and two subjects received docetaxel dose reductions. Eleven deaths occurred during the study. Eight of these were due to disease progression, two were due to AEs, and one was due to unknown causes. Of the two AEs leading to death, one (DIC) was considered possibly related to treatment, while the other (intracranial hemorrhage) was considered unrelated.

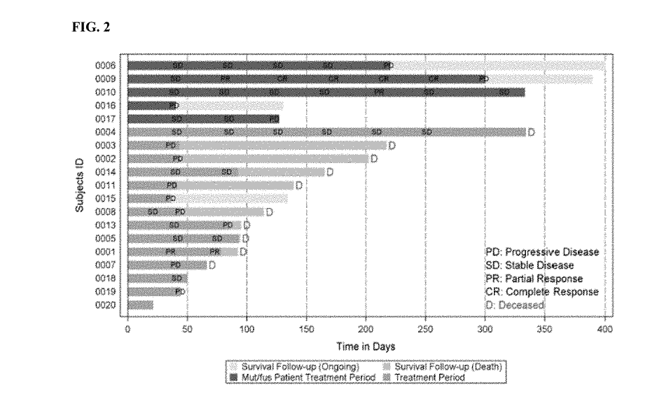

Overall, the combination of B-701 and docetaxel was found to be well tolerated, and produced an increase in ORR, DCR, PFS, and median OS in subjects with FGFR3 mutations/fusions. These results are summarized in Table 4 and FIG. 2.

TABLE 4

| | All subjects | FGFR3 mutation/ |
|---|---|---|
| | (n = 19) | fusion (n = 5) |
| Best response | 1 complete response | 1 CR, 1 PR |
| | (CR), 2 partial responses | |
| | (PRs) | |
| DOR | Not reached (NR) | NR |
| DCR | 58% | 80% |
| | (95% CI: 33.5-79.75) | (95% CI: 35.75-82.7) |
| PFS (months) | 3.25 | 7.9 |
| | (95% CI: 1.87-5.68) | (95% CI: 1.87-NR) |
| Median OS (months) | 5.68 | NR |
| | (95% CI: 3.25-11.3) | |

Additional clinical studies will be performed to further evaluate the effects of B-701 in combination with docetaxel or other taxanes in subjects with cancer. For example, a clinical study may be performed in which subjects with advanced or metastatic UCC will be randomized to receive either B-701 plus docetaxel or the current standard of care (e.g., docetaxel alone). Efficacy will be evaluated by PFS, as well as one or more additional parameters such as ORR, DCR, DOR, OS, AEs, or quality of life (QOL).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Bai et al. Cancer Res 70:7630 (2010)
2. Bumbaca et al. MAbs 3:376 (2011)
3. Brooks et al. Clin Cancer Res 18:1855-1862 (2012)
4. Lafitte et al. Mol Cancer 12:83 (2013)
5. Du et al. AACR-NCI-EORTC C63 (2011)
6. Zhao et al. Clin Cancer Res 16:5750 (2010)

What is claimed is:

1. A method of treating cancer in a subject in need thereof comprising administering a therapeutically effective amount of an FGFR3 inhibitor in combination with a therapeutically effective amount of a taxane.

2. The method of claim 1, wherein the FGFR3 inhibitor is an antagonistic FGFR3 antibody.

3. The method of claim 2, wherein the antagonistic FGFR3 antibody comprises CDR-H1 comprising the amino acid sequence set forth in SEQ ID NO:1, CDR-H2 comprising the amino acid sequence set forth in SEQ ID NO:2, and CDR-H3 comprising the amino acid sequence set forth in SEQ ID NO:3.

4. The method of claim 3, wherein the antagonistic FGFR3 antibody comprises a heavy chain variable region comprising the amino acid sequence set forth in SEQ ID NO:7.

5. The method of claim 2, wherein the antagonistic FGFR3 antibody comprises CDR-L1 comprising the amino acid sequence set forth in SEQ ID NO:4, CDR-L2 comprising the amino acid sequence set forth in SEQ ID NO:5, and CDR-L3 comprising the amino acid sequence set forth in SEQ ID NO:6.

6. The method of claim 5, wherein the antagonistic FGFR3 antibody comprises a light chain variable region comprising the amino acid sequence set forth in SEQ ID NO:8.

7. The method of claim 1, wherein the taxane is paclitaxel or an analog or prodrug thereof.

8. The method of claim 7, wherein the paclitaxel analog is selected from the group consisting of docetaxel and cabazitaxel.

9. The method of claim 1, wherein the cancer is a solid cancer.

10. The method of claim 9, wherein the solid cancer is selected from the group consisting of urothelial cancer, non-small cell lung cancer (NSCLC), head and neck cancer, and glioblastoma.

11. The method of claim 9, wherein the solid cancer comprises a mutation in FGFR3.

12. The method of claim 9, wherein the solid cancer comprises a gene fusion in FGFR3.

13. The method of claim 11 or 12, wherein FGFR3 is activated by the mutation or gene fusion.

14. A pharmaceutical composition comprising an FGFR3 inhibitor and a taxane.

15. The composition of claim 14, further comprising a pharmaceutically acceptable carrier.

16. The composition of claim 14, wherein the FGFR3 inhibitor is an antagonistic FGFR3 antibody.

17. The composition of claim 16, wherein the antagonistic FGFR3 antibody comprises CDR-H1 comprising the amino acid sequence set forth in SEQ ID NO:1, CDR-H2 comprising the amino acid sequence set forth in SEQ ID NO:2, and CDR-H3 comprising the amino acid sequence set forth in SEQ ID NO:3.

18. The composition of claim 17, wherein the antagonistic FGFR3 antibody comprises a heavy chain variable region comprising the amino acid sequence set forth in SEQ ID NO:7.

19. The composition of claim 16, wherein the antagonistic FGFR3 antibody comprises CDR-L1 comprising the amino acid sequence set forth in SEQ ID NO:4, CDR-L2 comprising the amino acid sequence set forth in SEQ ID NO:5, and CDR-L3 comprising the amino acid sequence set forth in SEQ ID NO:6.

20. The composition of claim 19, wherein the antagonistic FGFR3 antibody comprises a light chain variable region comprising the amino acid sequence set forth in SEQ ID NO:8.

21. The composition of claim 14, wherein the taxane is paclitaxel or an analog or prodrug thereof.

22. The composition of claim 21, wherein the paclitaxel analog is selected from the group consisting of docetaxel and cabazitaxel.
